(19) United States
(12) Patent Application Publication
(10) Pub. No.: US 2023/0372894 A1
(43) Pub. Date: Nov. 23, 2023

(54) DISPOSABLE ABSORBENT ARTICLE WITH ODOR REDUCING AGENTS

(71) Applicant: Freestyle World, Inc., Santa Monica, CA (US)

(72) Inventor: Jordan Frank, La Habra Heights, CA (US)

(73) Assignee: Freestyle World, Inc., Santa Monica, CA (US)

(21) Appl. No.: 17/746,815

(22) Filed: May 17, 2022

(51) Int. Cl. B01J 20/26; B01J 20/24; B01J 20/20; B01J 20/28; A61L 15/46; A61L 15/60; A61L 15/40

(52) U.S. Cl. CPC B01J 20/261; B01J 20/24; B01J 20/20; B01J 20/28038; B01J 20/28035; A61L 15/46; A61L 15/60; A61L 15/40

(57) ABSTRACT

An absorbent article, such as a diaper, disposable diapers, children's training pants, adolescent nighttime pants, feminine hygiene products, sanitary napkin or incontinence device, wherein said absorbent article comprising a top sheet, an acquisition-distribution layer, an absorbent core, and a back sheet, and where the absorbent core is layered with a wrapsheet, and the absorbent core further contains cellulosic material, an absorbent polymer and an odor reducing agent.

### FIELD OF THE ART

The present invention relates to the field of disposable highly absorbent wearable articles. These highly absorbent articles are designed to assist in avoiding leakage of bodily fluids and semi-fluids, such as urine, menses and related compositions. Materials, compositions and methods have been employed to reduce and hopefully eliminate the odor associated with these types of absorbent articles, but advances and improvements are always beneficial to the consumer and marketplace.

### BACKGROUND ART

Many odor reducing absorbent materials and articles are known in the field of disposable wearable absorbent articles. Common examples of absorbent articles include diapers, diaper covers, disposable diapers, children's training pants, adolescent nighttime pants, feminine hygiene products, sanitary napkin or incontinence device. Wearable absorbent articles are generally comprised of a top sheet, a back sheet, and an absorbent core. The back sheet is generally made from non-permeable materials, while the absorbent core is generally made of a combination of cellulosic fibers and absorbent materials. The absorbent materials can be superabsorbent materials. Absorbent article can further comprise additional materials to improve overall absorbency.

Additional materials can be employed in the absorbent article to improve the health of the wearer and/or odor issues associated with bodily fluids. Odors are the result of degradation of chemicals in the urine, and/or menses either trapped in the absorbent layer or held by the absorbent article. Additionally, urine, and menses contain microorganisms that are capable of producing odors and skin irritations. Components useful in treating/alleviating skin diseases, such as rash, dermatitis, or redness can include clay minerals, silicon oxide compounds, activated charcoal, or enzyme inhibitors. Additionally, components designed to improve or mask the odors associated with absorbent articles can be added to the top or back sheets. Known odor reducing components include urease inhibitors, enzymes, hydrogen peroxide, charcoal, and biocides. Unfortunately, while well intended, many of these odor reducing or skin care additives may have deleterious side effects causing potentially worsening skin diseases and/or hormonal disturbances in the wearer.

U.S. Pat. No. 8,217,220 describes an absorbent article comprising a film with a monomolecular layer of a polymer with a functional group and an active agent. The film is applied in a layer-by-layer deposition method in which the first polymer has a functional group and the second polymer has a second functional group capable of interacting with the first functional group. The combination of layered technology and reactive functional groups is designed to assist in the reduction of odor causing chemicals.

U.S. Pat. No. 8,574,683 describes absorbent materials employing the use of liquid carriers, and preferably hydrophobic liquid carriers comprising odor controlling agents in combination with cellulosic fibers. The hydrophobic liquid carriers were believed to overcome issues related to excess wetting and reduced absorbency of the article where surfactants were employed as the carrier for the odor reducing agent.

U.S. Pat. No. 10,391,004 describes a compostable diaper comprising biodegradable materials that assist in speeding up the breakdown of the diaper to reduce landfill volume and improve general waste disposal issues related to disposable diapers. A charcoal material is employed to expedite the breakdown of materials in the disposable diaper by increasing the microbial content of the diaper thereby increasing microbial development and facilitating the breakdown of the diaper. Separate biodegradable layers of non-woven material are stacked between a composting-stimulating layer containing charcoal. Alternatively, a charcoal containing layer can be applied to the non-woven materials.

U.S. Pat. No. 11,065,165 describes a multilayered disposable child sanitary napkin comprising a waterproof nonporous outer layer, a superabsorbent polymer layer, an odor absorbing activated charcoal pad layer, and a soft cotton covering layer. It further employs an imprinted product barcode and an embedded RFID chip allowing for tracking the child by a cellular connection.

U.S. Published Application No. 20040121681A1 describes an absorbent article made with an activated charcoal coating applied directly to a woven or nonwoven substrate. The activated charcoal coating is located outside the absorbent core and is designed to interact with human fluids as they pass through the substrate and into the absorbent material.

U.S. Published Application No. 2015/0190543 describes a superabsorbent hydrophilic three-dimensional fibrous web comprising odor reducing agents and super absorbent polymers in a complex and highly organized superstructure. While likely efficient in absorption, these structures are expensive and difficult to produce in large quantities.

Canadian Patent No. 2624278A1 describes an absorbent article that employs starch-based odor reducing agents. The starch-based odor reducing agents have specific particle size and surface area properties that allow the starch to form complexes in water thereby providing increased odor trapping ability.

U.S. Published Application No. 2008/0147029 describes an absorbent article, such as a diaper, containing a chemical compound having a positive charge as the odor controlling agent. More specifically, aluminum chlorohydrate is employed to immobilize bacteria that cause odors, thereby rendering the bacteria incapable of multiplying and producing additional odor causing sub stances.

The above descriptions of certain advantages and disadvantages of known odor-reducing agents for use in absorbent articles, and methods of their preparation, is not intended to limit the scope of the present invention. The present invention may include some or all of the methods and materials described above without the attendant disadvantages.

### SUMMARY OF THE INVENTION

In some embodiments the present invention is an absorbent article intended for use as a disposable diaper for children or as an incontinence device for adolescents or adults. The absorbent article will comprise a top sheet, an absorbent core and a back sheet. The top sheet will generally be a water permeable material that is gentle and non-irritating to the wearer. The absorbent core will generally be comprised of an absorbent polymer and cellulosic materials. A wrapsheet is located between the top sheet and the absorbent core. In certain embodiments, the wrapsheet surrounds the absorbent core. The polymers in the absorbent core are preferably comprised of super absorbent polymers. In certain embodiments, distributed within the absorbent core will be odor reducing agent, such as charcoal and/or activated charcoal. The back sheet is located below the absorbent core, and will generally be prepared from nonwoven water impermeable and/or breathable materials.

Embodiments of the present invention will include an acquisition-distribution layer between the top sheet and the absorption material. The acquisition-distribution layer enhances the uptake of liquid and semi-liquid components into the absorbent core.

Further embodiments of the present invention will include a back sheet that is comprised of an inner layer prepared from a breathable material, and an outer layer prepared from a polymeric film. The back sheet is located below the absorbent core and forms the exterior of the absorbent article. The breathable material provides moisture control and increases user comfort.

Embodiments of the present invention comprise wood pulp and mixtures of wood pulp and other cellulosic materials as the absorbent material in the absorbent core. Further the absorbent core contains odor reducing agents, such as activated charcoal admixed in, or distributed with, the wood pulp in the absorbent core.

Prior art absorbent articles are generally designed to absorb liquids and trap solids until the product can be disposed. Further, the use of sprayed-on fragrances and perfumes have been used to mask the scent of urine and other odors. Recently, the use of perfumes and fragrances have fallen out of favor due to the likelihood of unintended developmental side effects in children. This has led to an increase in the use of “fragrance-free” labeling of absorbent articles as a value-added feature. Existing methods for reducing odor in absorbent articles leave much to be desired. In contrast to the prior art products and methods, the present invention employs odor reducing agents present within the absorbent core to trap odors instead of covering or masking the unpleasant odor. The present invention provides the odor reducing agents within the absorbent core to separate the odor reducing agents from the skin of the wearer.

Odor reducing absorbent articles can be difficult and expensive to manufacture. The present invention provides advantages over the known methods and materials by utilizing the absorbent core as a substrate to contain the odor reducing materials. For example, U.S. Pat. No. 8,574,683 employs an additional liquid substrate to be added to the incontinence product. Additionally, U.S. Pub. App. No. 2004/0121681 describes an absorbent article with an activated carbon coating applied to a preformed substrate and heated to 100° C. to 300° C., which requires additional and costly processing steps. The present invention overcomes the above-described drawbacks by randomly depositing the odor reducing material directly into the absorbent core. This allows for a reduction in the processing steps as compared to the prior art, and a reduced need for additional carriers or substrate materials. Further, anti-odor or masking components, such as perfumes or additives, are eliminated from the absorbent article resulting in reduced costs, easier production, and improvements in usability. The usability is improved in many ways, one of which being a reduction in unwanted side effects and interactions with the perfume and additives.

The absorbent article of the present invention will comprise: a top sheet, an absorbent core, and a back sheet, wherein said top sheet is in contact with a wearer, below said top sheet is an acquisition-distribution layer, and below said acquisition-distribution layer is a wrapsheet which is in contact with said absorbent core; below said absorbent core is a back sheet, said absorbent core further comprises a cellulosic material, an absorbent polymer, and an odor reducing agent; wherein said cellulosic material is wholly or partially made from bamboo, said absorbent polymer is a super absorbent polymer and said odor reducing agent is activated charcoal; wherein the weight ratio of the activated charcoal to the super absorbent polymer is approximately 1:5 to 1:50; and wherein said activated charcoal is distributed in a non-uniform or random manner throughout the absorbent core.

### BRIEF DESCRIPTION OF THE DRAWINGS

FIG. 1 is a cross-sectional view of the absorbent article of the present invention.

FIG. 2 is an interior view of the absorbent core

FIG. 3 is an exploded view of the absorbent article of the present invention.

FIG. 4 is an embodiment of an absorbent article of the present invention.

### DETAILED DESCRIPTION OF THE INVENTION

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

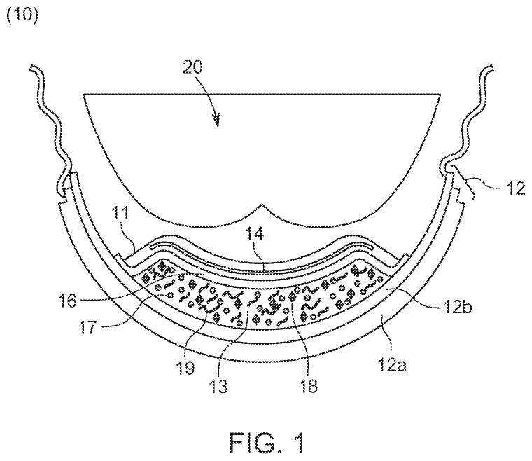

Turning now to FIG. 1, an absorbent article (10) designed for use as a disposable diaper for a child is shown having a top sheet (11). The top sheet (11) engages the skin of the wearer (20). Below the top sheet (11) is an acquisition-distribution layer (14). The absorbent core (13) is located between the acquisition-distribution layer (14) and a back sheet (12). The back sheet (12) may be prepared from two separate and combined layers, an outer layer that is a clothlike woven or non-woven material (12a), and an inner layer that is a breathable film layer (12b).

The absorbent article (10) further comprises a wrapsheet (16) between the acquisition-distribution layer (14) and the absorbent core (13). Additionally, located within the absorbent core (13) are absorbent polymers (17), odor reducing agents (18) and cellulosic materials (19). The term absorbent article as used in the present invention includes, but it is not limited to, diapers, disposable diapers, absorbent inserts for reusable diapers, children's training pants, adolescent nighttime pants, feminine hygiene products, sanitary napkin or incontinence device. The absorbent articles are intended to be discarded or partially discarded after a single use. The absorbent article may comprise a single inseparable structure, or replaceable inserts.

The top sheet of the present invention is generally prepared from nonwoven materials and is a hydrophilic and permeable material. The hydrophilic nature of the top sheet helps to reduce wetness to the user and avoid liquid run off out the absorbent article. It allows the liquid waste to pass through the top sheet into the absorbent core. Certain embodiments of the top sheet can be provided with holes to assist with the transport of the liquid into the absorbent core.

Examples of materials suitable for the top sheet include, but are not limited to, nonwoven fabrics often formed by processes such as meltblowing, spunbonding, solvent spinning, electrospinning, and carding. The basis weight of nonwoven fabrics is usually expressed in grams per square meter (g/m²). U.S. Pub. App. No. 2012/0316532, which is incorporated herein by reference, provides additional examples of materials suitable for top sheets according to the present invention. The preferred top sheet material is prepared from a combination of polyethene, polypropylene and polyethylene terephthalate. The preferred top sheet material has a density of 10 to 100 g/m², preferably a density of 25 to 70 g/m², and most preferably a density of at least 38 g/m².

The acquisition-distribution layer assists with the fluid management in the absorbent article. The acquisition-distribution layer is prepared from a hydrophilic material. It aids in the wicking of liquids from the user's skin to the absorbent core. The acquisition-distribution layer possesses an open structure and high bulk which provides for rapid liquid distribution and absorption in the absorbent core. Examples of materials suitable for the acquisition-distribution layer include, but are not limited to cellulosic materials cross-linked with dimethyl dihydroxyethylene urea or alkane polycarboxylic acids, and polyethylene/polypropylene bicomponent fibers. Additional examples can be found in U.S. Pat. No. 5,558,655, WO95/01147, EP 0 640 330 A1, and/or EP 0 631 768 A1, which are incorporated herein by reference. The preferred material for the acquisition-distribution layer is a combination of polyethylene and polypropylene, also known as a bicomponent fiber. The acquisition-distribution layer has a density of 10 to 90 g/m², preferably a density of 20 to 60 g/m², and most preferably a density of at least 35 g/m².

The term absorbent core as used in the present invention is the portion of the absorbent article that absorbs and retains the liquid and semi-liquid bodily fluids. The absorbent core can be prepared from various liquid absorbent materials, which are often cellulosic materials. Examples include comminuted wood pulp, often referred to as “air felt” or “fluff,” tissue, tissue wraps, tissue laminates, creped cellulose wadding, melt blown polymers, chemically stiffened cellulosic fibers, modified cellulosic fibers, cross-linked cellulosic fibers, absorbent foams, absorbent sponges, super absorbent polymers, superabsorbent fibers, absorbent gelling materials, and combinations thereof.

The absorbent polymers of the present invention are preferably super absorbent polymers. Super absorbent polymers include polymeric material that is water-insoluble and water swellable. These polymers are capable of absorbing high volumes of fluid in relation to their weight and retain the liquid under pressure. Examples of preferred super absorbent polymers include, but are not limited to, acrylic acid copolymers, saponified acrylic acid ester-vinyl acetate copolymer, hydrolyzed starch-acrylonitrile graft copolymer, acrylamide copolymer, neutralized starch-acrylic acid graft copolymer, hydrolyzed acrylonitrile copolymer, modified cross-linked polyvinyl alcohol, neutralized self-cross-linking polyacrylic acid, cross-linked polyacrylate salt, carboxylated cellulose, cross-linked polyacrylates including sodium polyacrylate and polyacrylamides; graft copolymers on polysaccharides such as chitin, chitosan, cellulose, starch, natural gums and polypeptide-based copolymers such as saponified starch-graft polyacrylonitrile, hydroxypropyl-cellulose (HPC); hydroxypropylmethylcellulose (HPMC), carboxymethyl-cellulose (CMC); carboxymethyl starch (CMS); cross-linked copolymers of maleic anhydride; polyvinyl alcohol; polyvinyl ethers; polymers and copolymers of vinyl sulfonic acid; copolymer, and neutralized crosslinked isobutylene-maleic anhydride copolymer.

The super absorbent polymer can be prepared in various forms, including, particles, beads, agglomerations, granules, spheres, flakes, fibers, and amorphous particles. The preferred form of the super absorbent polymer is in agglomerations or granules.

The super absorbent polymers according to the present invention will be distributed throughout the absorbent core and be present at about 20-80% by weight of the absorbent core, preferably 30-60% by weight of the absorbent core, and most preferably about 40-55% by weight of the absorbent core. The most preferred super absorbent polymer is a combination of two commercial grade superabsorbent polymers wherein both are acrylic acid copolymers, or polymers of acrylic acid, such as those available from BASF (Hysorb N7059) and Sumitomo (Aqua Keep SA605).

Cellulosic fibers as used in the present invention refer to cellulosic fluff pulps that are capable of forming a web structure to aid in absorption. Examples of cellulosic fluff pulps are softwood pulp including, but not limited to, southern pine, western hemlock, white pine, Caribbean pine, spruce, Douglas fir; hard wood pulp including, but not limited to, gum, maple, oak, aspen eucalyptus, poplar, beech; cellulosic fiber including, but not limited to cotton linter, flax, bamboo, bagasse, grass and kemp; and combinations thereof. Preferably, the cellulosic fiber of the present invention is derived from bamboo. The cellulosic fiber according to the present invention will be present at about 10-90% by weight of the absorbent core, preferably 30-80% by weight of the absorbent core, and most preferably about 45-65% by weight of the absorbent core.

The cellulosic fibers may be pretreated prior to their use in the absorbent article. Examples of pretreatment include physical treatment such as steam, and chemical pretreatment, such as thermomechanical pulp treatment. The cellulosic fibers can be obtained and processed for use in the present invention in the form of cellulose fibers in sheet, roll, or fluff form. Further, the cellulosic material can be provided as a mat of nonwoven material, for example, as a thermal bonded non-woven mat, or a stabilized resin-bonded material. In a common application, a sheet of cellulosic material is pulverized in a hammermill or fiberizer to create distinct and shorter fibers. These short fibers are combined with superabsorbent polymers through gravity, vacuum, or positive air pressure to form the absorbent core.

The absorbent core can be layered with or surrounded by a wrapsheet. The wrapsheet can be prepared from hydrophilic water permeable materials, preferably nonwovens, such as the ones described for the top sheet of the present invention. The wrapsheet aids in the liquid permeation into the absorbent core. The wrapsheet additionally provides support and structure to the absorbent core securing it within the absorbent article. The wrapsheet also contains the loose particles and fibers of the absorbent core inside the absorbent article. U.S. Pub. App. No. US20080147029, which is incorporated herein by reference, provides additional examples of wrapsheet materials that can be employed with the present invention. In one embodiment the wrapsheet is prepared from a spunbond polypropylene. In a preferred embodiment the wrapsheet is prepared by combining a first material that is a combination of cellulosic material (wood pulp) and polypropylene with a second material prepared from polypropylene. The first layer of the wrapsheet has a density of 10 to 120 g/m², preferably a density of 30 to 70 g/m², and most preferably a density of at least 45 g/m². The second layer of the wrapsheet has a density of 1 to 30 g/m², preferably a density of 5 to 20 g/m², and most preferably a density of at least 12 g/m².

The back sheet is designed to prevent waste material either absorbed into the absorbent core or contained within the absorbent article from escaping. The back sheet is generally substantially water-impermeable and prepared from breathable materials that will permit gases and vapors to escape from the absorbent article, but substantially stop waste products from traversing out of the absorbent article. Preferred breathable materials suitable for the back sheet include woven webs, nonwoven webs, composite materials such as film-coated nonwoven webs, apertured formed films and microporous films. Composite materials comprising polymer blends are also suitable for the back sheet. Additional examples can be found in WO 95/16746, U.S. Pat. Nos. 5,865,823, 5,571,096, and 6,107,537 which are incorporated herein by reference.

Back sheets can be prepared by multiple different techniques, such as surface treatments, film selection and processing, and/or particular filament selections and processing. Additionally, the back sheet can be prepared in multiple layers, with an outer layer and an inner layer, wherein the inner layer can be prepared from breathable soft non-woven material and the outer layer can be prepared from a substantially liquid-impermeable film. The layers can be prepared by known techniques, such as the use of adhesives or other bonding methods.

The back sheet of the present invention is generally prepared from nonwoven materials. The nonwoven materials suitable for the present invention include, but are not limited to, plastic films, polymeric materials such as apertured plastic films, apertured formed thermoplastic films, hydroformed thermoplastic films, reticulated thermoplastic films, and thermoplastic scrims; porous foams; reticulated foams; synthetic fibers, such as polymeric fibers including polyesters, polypropylene or polyethylene fibers, or from a combination of natural and synthetic fibers; nonwoven materials coated with water impermeable materials, and/or hydrophobic nonwoven materials, and combinations of the preceding materials often bonded together. The breathable material for use in the back sheet of the present invention allows additional airflow to the absorbent article assisting in water absorption and distribution of odorous gasses. Additional examples of breathable materials that are also breathable films include, but are not limited to porous polymeric films, nonwoven fabrics from spundown, hydrophobic and hydrophilic web or foam materials, tissue, and meltdown fibers. Embodiments wherein the back sheet comprises a nonwoven web can be prepared by various known techniques, including, but not limited to spun-bonded, melt-blown, hydroentangled, carded, wet-laid, or combinations thereof.

The preferred material for the back sheet is a two-layer preparation, wherein the outer layer is a water impermeable nonwoven polypropylene material, and the inner layer is a breathable nonwoven polyethylene material Preferably the back sheet has a density or basis weight of 5-50 g/m², preferably a basis weight of 10-25 g/m², and most preferably a basis weight of 14 g/m².

Skincare agents useful in the present invention offset the irritating components in urine or excrements. Examples of useful skincare agents are clay minerals, such as montmorillonite, bentonite, kaolin; silicon oxide compounds, such as zeolites, quartz, activated charcoal; enzyme inhibitors, such as metal salts of iron or zinc, metal ions such as copper or silver; ethylene diamine tetraacetic acid (EDTA), soybean trypsin inhibitor, stearyl glycyrrhetinate, glycerol triacetate, lima bean protease inhibitor, maize protease inhibitor, betaine compounds, sulphobetaine compounds, cholestyramine, p-guanidinobenzoates; and combinations thereof.

The absorbent article of the present invention may also contain a wetness indicator. The purpose of the wetness indicator is to inform a caregiver that the diaper has been exposed to liquid waste and should be changed. Wetness indicators can be prepared from a colorant that reacts when in contact with liquids such as urine, liquid solid waste, and/or menses. Preferably, the color change should be highly distinctive and remain for an extended period of time so that the caregiver is aware of the need to change the diaper. The wetness indicator can be printed, adhered to, or embedded into the back sheet of the absorbent article so that it is viewable to the caregiver while the absorbent article is in use. The absorbent article can contain multiple wetness indicators located at multiple locations. The wetness indicator can be prepared from colorants, pH sensitive materials, glues, printable inks, and other materials that will react to liquid, and are safe and compatible with the absorbent article of the present invention. The preferred wetness indicator is a glue prepared by Henkel (9817U). The wetness indicator is present in the absorbent article at a weight percent of the of about 0.01 to 2% of the absorbent article, and most preferably 0.1 to 1% of the absorbent article.

The present invention employs an odor reducing agent to sequester and/or deactivate the odors produced by urine, menses and/or other bodily fluids trapped in the absorbent core, or the bacteria contained in said materials. Unpleasant odors are generally produced by the degradation of organic compounds such as urine or menses. Aromatic ammonium compounds are produced by the breakdown of urea in urine. Attempts have been made to mask these odors with perfumes and covering agents, but it has been found that such compounds can cause deleterious effects to the wearer's skin and or endocrine system. An improvement provided by the present invention is the use of odor reducing agents located randomly or non-uniformly throughout the absorbent core. The random distribution of odor reducing agents serves multiple purposes, including, but not limited to trapping and deactivating aromatic compounds, and immobilizing bacteria. Reducing the bacteria's ability to multiply inhibits and destroys its ability to produce odor. Useful odor reducing agents according to the present invention include, but are not limited to, urease inhibitors, enzymes, cyclodextrins, hydrogen peroxide, biocides, zeolites, and carbon compounds. Additional odor reducing agents are starch based compounds such as amylose, such as V-amylose, activated starch and linear dextrin, such as those described in CA2624278A1, which is incorporated herein by reference. These starch-based compounds are relatively moisture insensitive and provide enhanced surface area for odor reduction. Zeolites that can be employed in the present invention include, but are not limited to analcime, chabazite, clinoptilolite, heulandite, natrolite, phillipsite, and stilbite, and combinations thereof.

The preferred odor reducing agent of the present invention is a carbon compound, more specifically, charcoal, and most preferred is activated charcoal. Activated charcoal, also referred to as activated carbon or AC, is a processed form of carbon with small low-volume pores which increase the surface area available for adsorption or chemical reactions. One gram of activated charcoal has a surface area in excess of 3,000 m² (32,000 sq ft). Chemical treatment can additionally enhance the adsorption properties of activated charcoal.

Studies have shown that activated carbons can exhibit antibacterial properties. Thamke et al., Comparison of Bacterial Contamination and Antibacterial Efficacy in Bristles of Charcoal Toothbrushes versus Noncharcoal Toothbrushes: A Microbiological Study, Contemp. Clin. Dent., 9(3):463-467 (2018) demonstrated the improvements in reducing bacterial contamination when using activated charcoal toothbrush bristles. Abeysinghe et al., Evaluation of antibacterial activity of different mangrove plant extracts, Ruhuna J. Sci., Vol. 1, September, pp. 104-112 (2006) shows that leaf extracts treated with charcoal exhibited increased bacterial inhibition. Osmokrovic et al., Achieving high antimicrobial activity: Composite alginate hydrogel beads releasing activated charcoal with an immobilized active agent, Carbohydrate Polymers, Vol. 196, September, pp. 279-288 (2018) demonstrated that polymeric beads containing activated charcoal provided antibacterial efficacy. By adding activated charcoal to the absorbent core bacterial growth can be reduced while the absorbent article is in use. Additionally, bacterial growth can further be reduced after disposal of the absorbent article.

The odor reducing agent is located at intervals throughout the absorbent core. In a preferred embodiment the odor reducing agent is dispersed randomly in the absorbent core amongst the super absorbent polymers. Alternatively, the odor reducing agent is dispersed at specific locations throughout the absorbent core. In another embodiment the odor reducing agent is deposited on the top or bottom surfaces of the absorbent core. In a further embodiment the odor reducing agent is deposited along with the pulp fiber (cellulosic material) and milled along with the pulp. The odor reducing agent according to the present invention will be about 0.5-10% by weight of the absorbent core, preferably 1-7% by weight of the absorbent core, and most preferably about 3-6% by weight of the absorbent core.

The activated charcoal can be prepared from a wide variety of materials, including coal, coconut shells, hard woods, softwoods, bamboo, peat, petroleum coke, sawdust or combinations thereof. Methods of preparing the activated charcoal from the source material are generally known. The activated charcoal of the present invention can be provided in various forms and sizes. Preferably, the activated charcoal is granulated, and most preferably micronized. Suitable particle sizes for the activated charcoal of the present invention can range from 0.2 mm to 5 mm, preferably 1 mm to 2 mm. In pelletized form the activated charcoal can have a median diameter of about 0.8 to 5 mm, and preferably 1 mm to 2 mm. In powdered form the activated charcoal can have a median particle size of 15 to 500 microns, preferably 270 to 400 microns.

The activated charcoal is randomly distributed throughout the absorbent core of the present invention. Additionally, the activated charcoal is present at a weight ratio from 1:10 to 1:1000 in relational to the total weight of the absorbent core. More preferably, the activated charcoal is present at a weight ratio of 1:1 to 1:250 in relational to the total weight of the absorbent core, and most preferably 1:18 to 1:33. The weight ratio of activated charcoal to the super absorbent polymers in the absorbent core is from 1:5 to 1:200, and more preferably 1:7.5 to 1:15. The weight ratio of activated charcoal to the cellulosic material in the absorbent core is from 1:5 to 1:250, and more preferably 1:8 to 1:30, and most preferably 1:10 to 1:17.

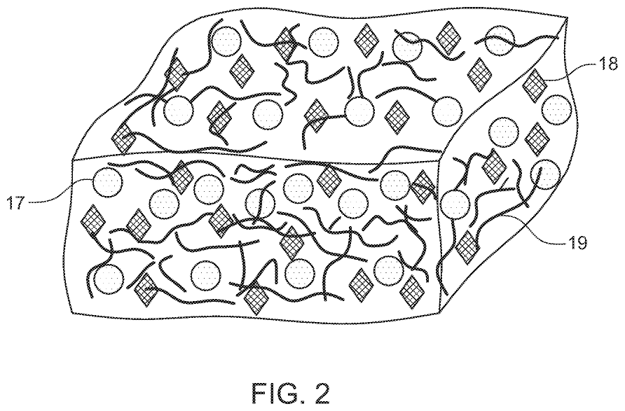

FIG. 2 shows an interior view of an embodiment of the absorbent core (13) of the present invention. The absorbent core (13) contains cellulosic material (19) embedded with absorbent polymers (17) and odor reducing agents (18). The absorbent polymers (17) are preferably super absorbent polymer and the odor reducing agents (18) are preferably activated charcoal. The super absorbent polymers are generally distributed in a uniform manner throughout the absorbent core (13), while the activated charcoal is generally distributed in a random or non-uniform manner throughout the absorbent core (13).

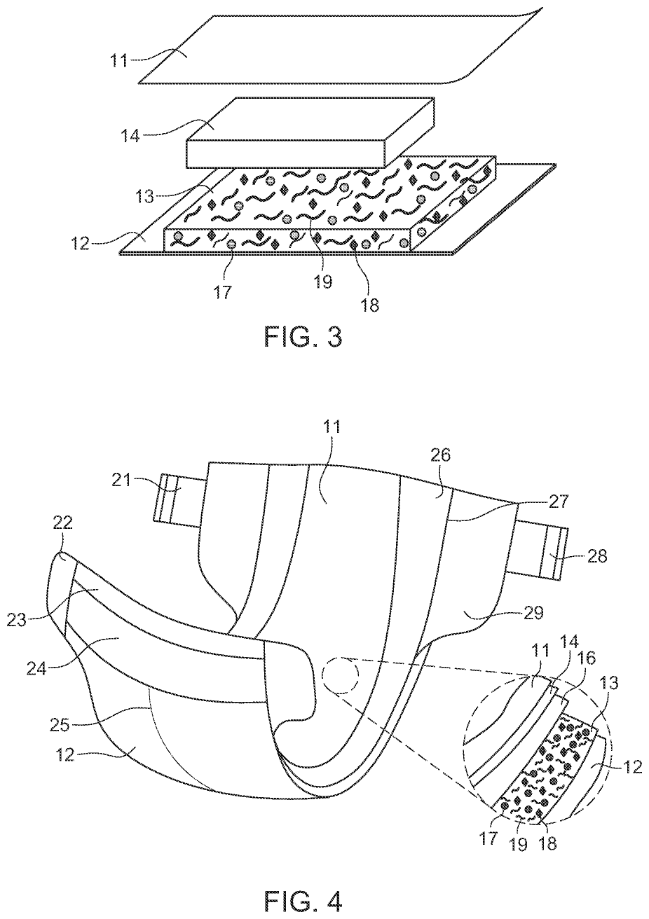

FIG. 3 is an exploded view of the core structure of the absorbent article (10). Specifically, FIG. 3 shows the top sheet (11) above the acquisition-distribution layer (14). The absorbent core (13) is located below the acquisition-distribution layer (14). The back sheet (12) is located below the absorbent core (13). Additionally, located within the absorbent core (13) are absorbent polymers (17), cellulosic materials (19), and odor reducing agents (18).

FIG. 4 shows an embodiment of a diaper according to the present invention, with a highlighted cross section of the core of the diaper according to the present invention. Specifically, the diaper represented in FIG. 4 shows the location of additional components of a commercial diaper product as commonly employed in the art. It is well known in the industry how to manufacture and apply these additional components to prepare a finished diaper product. The following additional components are shown in FIG. 4: fastener tape (21), front-ears (22), umbilical notches (23), tape fastening zones (24), wetness indicators (25), inner cuff (26), elastic (27), fastening hooks (28), and back-ears (29), are applied to the prepared materials by processes commonly known in the art.

### Example 1

One embodiment of the present invention is manufactured according to the following steps:

The absorbent article of the present invention is prepared in a multistep process. First the layered materials are selected and prepared for processing. The top layer is prepared from a combination of polyethylene, polypropylene and polyethylene terephthalate with a weight basis (or density) of 38 g/m². Next the acquisition distribution layer is prepared from a combination of polyethylene and polypropylene, which is a bicomponent fiber. The acquisition-distribution layer has a weight basis (or density) of 35 g/m². The wrapsheet is prepared by combining a first material that is a combination of cellulosic material (wood pulp) and polypropylene with a second material prepared from polypropylene. The first layer of the wrapsheet has a weight basis (density) of 45 g/m². The second layer of the wrapsheet has a weight basis (density) of 12 g/m². The back sheet is a two-layer preparation, wherein the outer layer is a water impermeable nonwoven polypropylene material, and the inner layer is a breathable nonwoven polyethylene material. The back sheet has a density or basis weight (density) 14-16 g/m².

The absorbent core is prepared from cellulosic fibers derived from bamboo. The bamboo pulp comprises approximately 50% of the weight of the absorbent core. The bamboo pulp is impregnated with two super absorbent polymers, both of which are polymers of acrylic acid (BASF (Hysorb N7059) and Sumitomo (Aqua Keep SA605)). The super absorbent polymers are present at about 50% of the weight of the absorbent core. Further the absorbent core contains activated charcoal as an odor reducing agent. The activated charcoal is present in the absorbent core at 3-5.4% by weight of the absorbent core. The activated charcoal is distributed randomly in a non-uniform manner throughout the absorbent core. Additionally, the particle size of activated charcoal is approximately 50-150 microns (325-100 mesh). The bamboo pulp, super absorbent polymers, activated charcoal are blended together to prepare the absorbent core material.

Processes and techniques for combining the topsheet, acquisition-distribution layer, absorbent core, wrapsheet, and backsheet are well known in the art. Further, the final commercial product includes additional components such at fastener tape, front-ears, umbilical notches, tape fastening zones, inner cuffs, elastic, fastening hooks, wetness indicators, and back-ears, all of which are applied to the prepared materials by processes and techniques commonly known in the art.

The foregoing description and examples have been set forth merely to illustrate the present invention and are not intended to be limiting. Since modifications of the described embodiments incorporating the spirit and substance of the invention may occur to persons skilled in the art, the invention should be construed broadly to include all variations within the scope of this application, including but not limited to the appended claims and equivalents thereof.

1. An absorbent article comprising a top sheet, an absorbent core, and a back sheet, wherein said top sheet is in contact with a wearer, below said top sheet is an acquisition-distribution layer, and below said acquisition-distribution layer is a wrapsheet which is in contact with said absorbent core; below said absorbent core is a back sheet, said absorbent core further comprising a cellulosic material, an absorbent polymer and an odor reducing agent.

2. The absorbent article according to claim 1, wherein said absorbent article is selected from the group consisting of diapers, disposable diapers, children's training pants, adolescent nighttime pants, feminine hygiene products, sanitary napkin or incontinence device.

3. The absorbent article according to claim 1, wherein said absorbent article is a disposable diaper.

4. The absorbent article according to claim 1, wherein said cellulosic material is bamboo pulp, said absorbent polymer is a super absorbent polymer and said odor reducing agent is activated charcoal.

5. The absorbent article according to claim 4 wherein said super absorbent polymer is selected from the group consisting of acrylic acid copolymers, saponified acrylic acid ester-vinyl acetate copolymer, hydrolyzed starch-acrylonitrile graft copolymer, acrylamide copolymer, neutralized starch-acrylic acid graft copolymer, hydrolyzed acrylonitrile copolymer, modified cross-linked polyvinyl alcohol, neutralized self-cross-linking polyacrylic acid, cross-linked polyacrylate salt, carboxylated cellulose, cross-linked polyacrylates including sodium polyacrylate and polyacrylamides; graft copolymers on polysaccharides such as chitin, chitosan, cellulose, starch, natural gums and polypeptide-based copolymers; saponified starch-graft polyacrylonitrile, hydroxypropyl-cellulose (HPC); hydroxypropylmethylcellulose (HPMC), carboxymethyl-cellulose (CMC); carboxymethyl starch (CMS); cross-linked copolymers of maleic anhydride; polyvinyl alcohol; polyvinyl ethers; polymers and copolymers of vinyl sulfonic acid; neutralized crosslinked isobutylene-maleic anhydride copolymer; and combinations thereof.

6. The absorbent article according to claim 5 wherein said super absorbent polymer is a combination of two acrylic acid copolymers.

7. The absorbent article according to claim 4, wherein said activated charcoal is distributed in a non-uniform manner throughout the absorbent core.

8. The absorbent article according to claim 4, wherein said activated charcoal is distributed on the surface of the absorbent core.

9. The absorbent article according to claim 8, wherein the weight ratio of said activated charcoal to said absorbent core is 1:5 to 1:250.

10. The absorbent article according to claim 9, wherein the weight ratio of said activated charcoal to said absorbent core is 1:18 to 1:33.

11. An absorbent core comprising super absorbent polymers, activated charcoal and cellulosic materials, wherein said activated charcoal is distributed in a non-uniform manner throughout said absorbent core.

12. The absorbent core according to claim 11 wherein the particle size of the activated charcoal is about 50 to 150 microns.

13. The absorbent core according to claim 12 wherein the weight ratio of said activated charcoal to said absorbent core is 1:5 to 1:250.

14. The absorbent core according to claim 12 wherein the weight ratio of said activated charcoal to said absorbent core is 1:18 to 1:33.

15. The absorbent core according to claim 12 wherein the weight ratio of said activated charcoal to said super absorbent polymer is 1:5 to 1:200.

16. The absorbent core according to claim 12 wherein the weight ratio of said activated charcoal to said super absorbent polymer is 1:7.5 to 1:15.

17. The absorbent article according to claim 12 wherein said super absorbent polymer is selected from the group consisting of acrylic acid copolymers, saponified acrylic acid ester-vinyl acetate copolymer, hydrolyzed starch-acrylonitrile graft copolymer, acrylamide copolymer, neutralized starch-acrylic acid graft copolymer, hydrolyzed acrylonitrile copolymer, modified cross-linked polyvinyl alcohol, neutralized self-cross-linking polyacrylic acid, cross-linked polyacrylate salt, carboxylated cellulose, cross-linked polyacrylates including sodium polyacrylate and polyacrylamides; graft copolymers on polysaccharides such as chitin, chitosan, cellulose, starch, natural gums and polypeptide-based copolymers; saponified starch-graft polyacrylonitrile, hydroxypropyl-cellulose (HPC); hydroxypropylmethylcellulose (HPMC), carboxymethyl-cellulose (CMC); carboxymethyl starch (CMS); cross-linked copolymers of maleic anhydride; polyvinyl alcohol; polyvinyl ethers; polymers and copolymers of vinyl sulfonic acid; neutralized crosslinked isobutylene-maleic anhydride copolymer; and combinations thereof.

18. The absorbent article according to claim 17 wherein said super absorbent polymer is a combination of two acrylic acid copolymers.

19. An absorbent article comprising a top sheet, an absorbent core, and a back sheet, wherein said top sheet is in contact with a wearer, below said top sheet is an acquisition-distribution layer, and below said acquisition-distribution layer is a wrapsheet which is in contact with said absorbent core; below said absorbent core is a back sheet, said absorbent core further comprising an absorbent polymer and an odor reducing agent; wherein said absorbent polymers are super absorbent polymers and wherein said odor reducing agent are activated charcoal; wherein the weight ratio of said activated charcoal to said super absorbent polymer is 1:18 to 1:33; and wherein said activated charcoal is distributed in a non-uniform manner throughout the absorbent core.

20. The absorbent article according to claim 19, wherein said activated charcoal is distributed on the surface of the absorbent core.
